(19) United States
(12) Patent Application Publication
(10) Pub. No.: US 2026/0144914 A1
(43) Pub. Date: May 28, 2026

(54) MINIMALLY INVASIVE METHODS FOR PLASTIC AND AESTHETIC FACIAL RESTORATION USING BONE DERIVED COMPOSITIONS

(71) Applicant: MUSCULOSKELETAL TRANSPLANT FOUNDATION, Edison, NJ (US)

(72) Inventor: Marc LONG, Monmouth Junction, NJ (US)

(21) Appl. No.: 19/402,447

(22) Filed: Nov. 26, 2025

Related U.S. Application Data
(60) Provisional application No. 63/725,775, filed on Nov. 27, 2024.

(51) Int. Cl. A61L 27/36; A61L 27/54; A61L 27/56

(52) U.S. Cl. CPC A61L 27/3608; A61L 27/3612; A61L 27/54; A61L 27/56; A61L 2400/06; A61L 2430/02; A61L 2430/40

(57) ABSTRACT

Methods are provided for aesthetic restoration and reconstruction of bone loss, deficit, defect, or a combination thereof. The methods include administration of one or more suitable bone derived compositions by a minimally invasive procedure such as injection using a needle, syringe, etc., placement in or through a small incision, or a combination thereof. The properties and characteristics of the bone derived composition, including without limitation, flowability, injectability, size, shape, and physical form, are limited only by the requirement that they are capable of administration by a minimally invasive technique. For example, these methods are effective for restoration of facial features, e.g., lost chin definition, jawline definition, bone around nasal lines, and bone around the upper and lower eye area. These methods are also effective for treatment of bone deficiency and loss in other body features such as shoulders and collarbones, pelvis, feet, ankles, hands, and wrists.

### CROSS-REFERENCE TO RELATED APPLICATIONS

The present application claims the benefit of U.S. Provisional Application No. 63/725,775, filed Nov. 27, 2024, the entire contents of which is hereby incorporated by reference herein.

### FIELD OF THE INVENTION

The invention described and contemplated herein relates to methods for reconstruction of age-related and other defects and deficits of facial features

### BACKGROUND

In our youth, facial features are generally defined and well contoured. However, as we age, the loss of soft tissue elasticity and volume allows soft tissues, such as muscles, adipose, and skin, to sag, droop, and fall downward due to gravity. For example, facial fat pads may decrease in volume and, for instance, fat pad volume deficits in the cheeks and the malar area may become more visible, causing a reduction in fullness around the face, all of which are typical effects of aging on facial features.

The medical aesthetic market seeks to address and repair many of the aforesaid signs and symptoms of aging by providing many types of soft tissue fillers, including sodium hyaluronates (HyAs), collagen biostimulants, and adipose-derived compositions. The intent and effect of administering such plastic and aesthetic reconstruction materials is primarily reconstruction, restoration, and revolumization of soft tissues, such as skin and adipose (e.g., fat and fat pads). For example, skin structure and volume may be rejuvenated, skin elasticity may be increased, wrinkles and fine lines may be reduced, and facial contouring may be corrected or improved.

While facial lines and wrinkles are common externally visible signs of aging, aging also occurs beneath the skin and at every layer of the facial structure. Accordingly, normal aging also involves changes to structure and volume of facial bones. Facial bones provide the foundation for muscle, fat-pads, and skin, which provides us with our unique facial features. A youthful bone structure typically includes full and high cheeks, defined brow bones, and less sunken eye areas. With age, we experience facial bone loss. This type of bone loss changes the dimensions and contour of our face, causing areas around our eyes to get larger, a decrease in the angle of our brow bone, and a less sculpted jawline. While bone loss is “under the skin”, it visibly affects the external appearance of the aging face, and restoring the original or younger contour of the bone structure may provide an externally visible or discernible effect since it would modify the foundation and tissue baseline of the face.

The acceptance of gender fluidity and/or reassignment and the related plastic reconstruction and aesthetic procedures have also created a need for new treatments to adjust body features. For instance, a more defined jawline may be wanted to create a more masculine appearance. Soft tissue corrections may not result in the optimum results where a more permanent adjustment of facial features by correction the foundational bone structure and shape may be desired.

Aesthetic treatment and solutions have focused on the reconstruction, restoration and revolumization of soft tissues, but there has been no common solution for restoring deficient bone structure, or repairing defects and loss of bone of the face due to aging or other conditions. While the treatment of soft tissues may provide improvements in appearance, more significant and permanent outcomes may be achieved by treating the underlying bone tissues and bone facial structure. Bone derived matrices and tissue forms have been administered to bone treatment sites in thousands of applications and procedures, some for more than 20 years, and even specifically for craniomaxillofacial applications in open surgical procedures. However, such treatments using bone derived matrices have focused on repairing acute and traumatic defects. However, there do not appear to be effective methods for selecting and using suitable bone derived matrices and tissue forms for aesthetic and plastic reconstructive applications without open surgery. The presently described and contemplated invention provides such methods for aesthetic and plastic reconstruction of bone and bone features of the face by using suitable bone derived matrices and tissue forms which facilitate administration by injection or minimally invasive incision.

### SUMMARY OF THE INVENTION

A method for treatment of a body feature having a condition comprising one or more of bone loss, bone deficit, and bone defect, the method comprising the steps of: selecting and obtaining one or more bone derived compositions; and administering the one or more bone derived compositions to a treatment site which is on, in, proximate to, or a combination thereof, the body feature.

The step of administering may comprise one or more of: implanting, injecting, positioning, reshaping followed by administering, the one or more bone derived compositions to the treatment site, by a minimally invasive procedure. For example, without limitation, the minimally invasive procedure may comprise injection using a delivery device, delivery in or through a small incision, or a combination thereof. In some embodiments, the minimally invasive procedure comprises injection using a delivery device selected from a needle, a syringe, and a cannula, and having a gauge number of about 20 or lower.

The body feature may, for example without limitation, comprise one or more of: a facial feature, a skull, a shoulder, a collarbone, a pelvis, a long bone, a foot, an ankle, a hand, and a wrist. When the body feature comprises a facial feature, it may for example, without limitation comprise: a jawline, a chin, a brow bone, a cheekbone, a malar area, a bone proximate nasal lines, a bone proximate upper eye area, a bone proximate lower eye area, or a combination thereof.

In some embodiments of the method, the treatment may comprise restoration, reconstruction, volumizing, or both, of one or more of: a size, a shape, a contour, and a volume, of the body feature being treated.

For example, but without limitation, the one or more bone derived compositions may comprise a physical form including: particles, powder, pieces, fibers, at least partially dehydrated, non-dehydrated, flowable, injectable, shapable, at least partially solidifying, preshaped, molded, porous, a paste, a putty, a gel, a suspension, a mixture, a monolithic piece, a mass of smaller forms, and combinations thereof. The physical form of the one or more bone derived compositions may comprise a monolithic piece, a mass of smaller physical forms agglomerated together into a single piece, or a combination thereof, which has an overall size and shape selected and formed, molded, at least partially solidified, or otherwise produced to be specific to the treatment site or body feature being treated.

In some embodiments, the one or more bone derived compositions comprise bone derived fibers. In some embodiments, the one or more bone derived compositions comprise a demineralized bone matrix (DBM) and are flowable and injectable. In some embodiments, the one or more bone derived compositions comprise bone derived fibers in a flowable, injectable, composition which further comprises a viscosity modifier comprising hyaluronic acid, derivatives of hyaluronic acid, or both.

### BRIEF DESCRIPTION OF THE FIGURES

The present invention will be further explained with reference to the attached drawings, wherein like structures are referred to by like numerals and/or letters throughout the several views. The drawings shown are not necessarily to scale, with emphasis instead generally being placed upon illustrating the principles of the present invention.

FIG. 1 provides an image of a face showing facial features having bone deficiencies and losses typically caused by aging and which are treatable by administration of bone derived compositions by minimally invasive techniques:

FIG. 2 is an image of a youthful face showing the “triangle of youth” formed by several prominent youthful facial features:

FIG. 3 is an image of a face affected by aging and showing the “pyramid of age” formed by several facial features having bone deficiencies and losses: and

FIG. 4 is the image of FIG. 1 which indicates regions typically suitable for administration of bone derived compositions according to the presently described and contemplated method for aesthetic restoration and reconstruction of age-related bone deficiencies and losses.

### DETAILED DESCRIPTION

The invention described and contemplated herein provides methods for aesthetic restoration and reconstruction of facial features having or including bone loss, deficit, defect, or a combination thereof. While primarily focused on restoration and reconstruction of facial bone, the presently described and contemplated invention is not so limited. As will be recognized and understood by persons of ordinary skill in the relevant art, the methods described and contemplated herein are also useful and effective for treatment of bone loss, deficit, defect, or a combination thereof, in other body features including, without limitation, shoulders and collarbones, long bones, pelvis, feet, ankles, hands, and wrists. It should be understood that other conditions involving bone deficiency or loss which may be treated or resolved without surgical intervention or intervention in an operating room (i.e., requiring administration of general anesthesia), will benefit from application of the methods described and contemplated herein. Patients treated without surgical intervention typically do not receive general anesthesia, but rather, local or partial anesthesia, or sedation, is typically administered.

It is noted that shifting from open procedures to minimally invasive procedures should allow decreased in-patient hospital admissions, i.e., shorter or even no in-patient stays. Providing effective minimally invasive methods for aesthetic restoration and reconstruction of body features, such as facial features (e.g., by preparation and/or selection of suitable bone-derived matrix graft materials and their administration to bone containing features of interest), may even enable moving such treatments from hospitals to outpatient surgical centers or to the offices of qualified providers (doctor, surgeon, etc.). It is further envisioned that the minimally invasive methods described and contemplated herein may further allow elimination of inhalation anesthesia (and associated risks), as well as decrease scarring with quicker healing, all of which enhance patient quality of life.

In an exemplary embodiment, a method for aesthetic restoration and reconstruction of body features, such as facial features, having or including bone loss, deficit, defect, or a combination thereof, comprises selecting one or more suitable bone derived compositions, and administering the selected one or more bone derived compositions to the body feature by a minimally invasive technique, which include without limitation, injection using a delivery device such as a needle, syringe, or cannula, delivery in or through a small incision, and combinations thereof. In some embodiments, prior to administering the selected on or more bone derived compositions, site preparation may be performed and may include debridement, excision, or other manipulation or modification, of tissue at or proximate the treatment site. Site preparation may, for example without limitation, be performed using a spatula cannula.

The biological source of the bone derived compositions is not particularly limited, but rather, includes mammalian (human and non-human), avian, amphibian, reptilian, and combinations thereof. In some embodiments, the source of the bone derived compositions includes one or more of human, bovine, ovine, equine, porcine, canine, etc. Likewise, the subject having a body feature, such as a facial feature, which has or includes bone loss, deficit, defect, or a combination thereof to be aesthetically restored and reconstructed is not particularly limited, but rather, may include any of several families and species such as human and non-human mammals, birds, amphibians, reptiles, etc.

Suitable bone derived compositions include those having properties enabling them to be administered or delivered to a treatment site by minimally invasive techniques (procedures). The treatment site will be in, on, proximate to, or a combination thereof, a body feature being treated. Suitable bone derived compositions may also have properties or characteristics which are beneficial, effective, or both, for restoration and reconstruction of the size, shape, contour, volume, etc. of the body feature being treated.

For example, properties which enable bone derived compositions to be administered or delivered to a treatment site by injection, using delivery device such as, but not limited to: a needle, a syringe, a cannula, or similar delivery device, include being flowable or injectable and, therefore, capable of passage and administration (delivery) through the delivery device. The ability of the bone derived composition to pass through a delivery device depends, at least in part, on the size (dimensions) of the bone derived composition which remains capable of promoting the restoration, reconstruction, or both, of a body feature involving bone loss, deficit, defect, or a combination thereof, as well as the amount and type of carrier if any that is combined therewith. For example, the bone derived composition should be capable of delivery through a delivery device having a gauge number of about 20 or lower, such as without limitation, 19 gauge or lower, or even 18 gauge, or lower. As understood by persons of ordinary skill in the relevant art, but stated here for confirmation, gauge numbers are inversely related to the size of the opening or inner diameter of the delivery device. The larger the gauge number of a delivery device, the smaller the opening or inner diameter of the device through which flowable material will passes for delivery.

Additionally, properties of bone derived compositions which enable them to be delivered in or through a small incision include being sized and shaped to fit through an incision of length from about 1 centimeter (cm) to about 0.5 millimeter (mm), or any length therebetween such as, without limitation, about 8 mm or less, or 5 mm or less, or even 2 mm or less. Accordingly, in some embodiments, one or more bone derived compositions, including but not limited to, flowable, injectable, shapable (e.g., in paste or putty form), preshaped, molded, or otherwise formed (e.g., at the time of production or at the time of use), are administered (implanted, injected, placed, positioned, reshaped and then implanted or positioned, etc.) to (on/in) a body feature (e.g., a bony facial feature) by a minimally invasive procedure. The bone derived compositions may retain their original form after implantation, or may solidify or partially solidify in-situ after injection.

The shape or physical form of suitable bone derived compositions is not particularly limited so long as it is capable of administration by a minimally invasive method as described and contemplated above. For example, without limitation, the bone derived composition may include: particles, fibers (e.g., elongated particles), powder, pieces, chips, flakes, cubes, strips, ribbons, threads, cones, wedges, irregular or asymmetric shapes, a single mass or shape, and combinations thereof. Suitable bone derived compositions may have a physical form including, but not limited to: a dehydrated form (e.g., particles, powder, pieces, fibers, etc.), a partially dehydrated form, a non-dehydrated form, a porous form, a paste, a putty, a gel, a suspension, a mixture, and combinations thereof.

In some embodiments, suitable bone derived compositions include a monolithic piece or mass of smaller physical forms agglomerated together into a single piece, which have an overall size and shape which is selected and may be formed, molded or otherwise produced or provided to be specific to application and the feature or site to be treated. In some embodiments, monolithic piece or mass of smaller physical forms may be porous, such as a porous sponge. For example, without limitation, the bone derived composition may comprise a piece of bone tissue which has been formed by any of various techniques (e.g., cutting, shaving, lathing, molding, etc.) into a three dimensional shape such as, but not limited to, a cube, brick, sphere, sheet, patch, or combination thereof, to produce a bone derived composition which is porous, with or without further shaping. In some embodiments, for example, without limitation, a porous cancellous bone derived block or piece may be produced, at least in part, by at least partially demineralizing a piece of bone, with reshaping performed before, after, or both before and after the demineralizing. The cancellous bone structure which remains after at least partially demineralizing a cancellous bone piece is porous and compressible. At least partially demineralized cortical bone pieces (e.g. derived from long bone samples) will be porous, flexible, and compressible.

Properties and characteristics of suitable bone derived compositions which may be beneficial, effective, or both, for restoration and reconstruction of a body feature having or including bone loss, deficit, defect, or a combination thereof, include without limitation: containing viable cells, absence of viable cells, containing non-viable cells and cell components, demineralized, partially or selectively demineralized, non-demineralized, containing one or more endogenous bioactive materials (e.g., proteins, growth factors, cytokines, vesicles, exosomes, extracellular matrix proteins, cells and cellular components, etc.), containing one or more exogenous materials and substances whether bioactive or not (e.g., cells and other bioactive materials, polymers, carriers, excipients, fillers, viscosity modifiers, antibiotics, anticoagulants, binders, etc.). Without limitation, viscosity modifiers may include hyaluronic acid and its derivatives (e.g., sodium hyaluronate, etc.), methylcellulose and its derivatives, and the like, and combinations thereof.

The processing steps performed to produce the one or more bone derived compositions are not particularly limited but may, without limitation, include any one or more of: cutting, shaving, milling, molding, and otherwise reshaping, cleaning, rinsing, soaking, at least partially decellularizing, at least partially demineralizing, disinfecting, sterilizing, and any other steps which produce the bone derived compositions in a desired form and condition (e.g., physical, structural, contaminant free, and bioactive or not, etc.). Furthermore, the bone derived compositions may be derived or produced from any type of bone tissue recovered from one or more donors including, but not limited to: cortical, cancellous, lamellar, and combinations thereof.

Furthermore, without limitation, the bone derived composition may or may not be combined (mixed, attached, connected, deposited, inserted, infused, layered, etc.) with other materials and compositions, which may or may not be tissue derived, endogenous, exogenous, bioactive, inert, natural, synthetic, etc. (as described above for exogenous materials and substances). For example, without limitation, the one or more bone-derived compositions may be combined other tissue derived materials such as, but not limited to, cartilage particles, cartilage fibers, other cartilage tissue forms, or a combination thereof, or placenta derived particles, sheets or patches, or with exogenous growth factors, or cells, and other materials as are known by persons of ordinary skill in the relevant art.

The methods described and contemplated herein are effective for aesthetic restoration and reconstruction of body features, such as facial features, having or including bone loss, deficit, defect, or combinations thereof, for example, as the result of aging or another condition or process. Accordingly, a contemplated application is for aesthetic reconstruction of the face and skull where bone loss is observed by administering a flowable demineralized bone fiber matrix (DBM), such as but not limited to demineralized bone fibers, particles, or both, combined with a carrier or other flowable or viscous material, or a preshaped or formed bone derived graft, to correct skull or jaw lines, or implanting a small, shaped piece of bone through a minimal (non-visible) incision.

This invention focuses on restoration, modification, and correction of bone loss and bone defects involving the boney foundation of faces (e.g., facial features) and skulls.

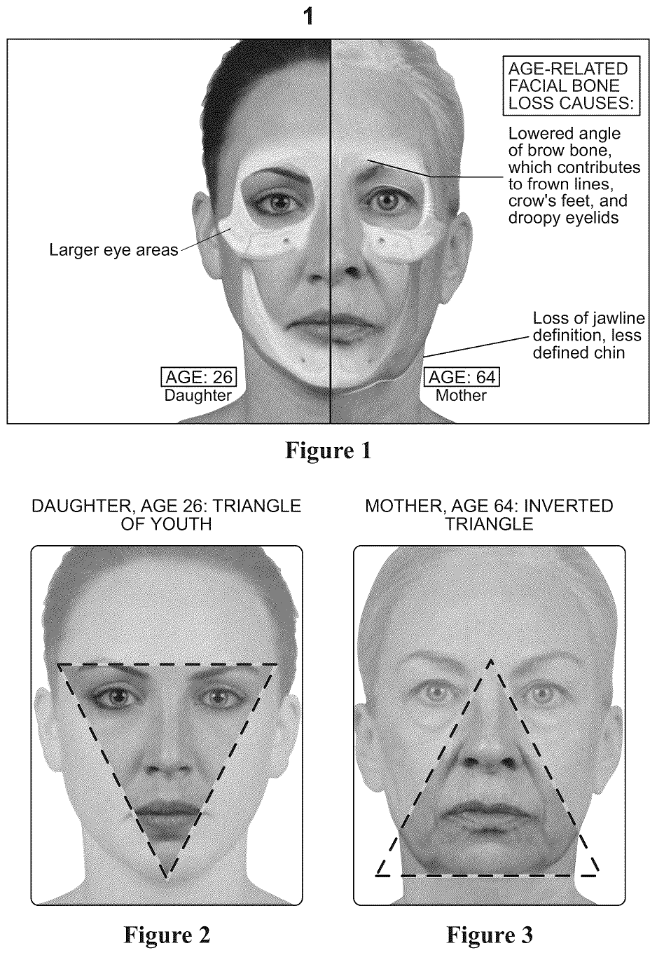

Facial bones provide the foundation for muscle, fat-pads, and skin. This bone structure is what gives us our unique facial shape and contour. A youthful bone structure has full and high cheeks—as shown in the triangle of youth—and defined brow bones and less sunken eye areas. With age, we experience facial bone loss. This type of bone loss changes the dimensions and contour of our face, causing areas around our eyes to get larger, a decrease in the angle of our brow bone, and a less sculpted jawline. The split-face image provided in FIG. 1 suggests facial features where the bone derived compositions may be administered according to the presently described and contemplated method to restore the facial bone structure and correct, fill or augment bone defects and volume losses typically resulting from aging.

While facial lines and wrinkles are common and visible signs of aging, aging effects also occur beneath the skin, and at every layer of the facial structure. In our youth, facial features are defined and well contoured, including without limitation such as high cheekbones, full cheeks, and defined jawline. The aforesaid collection of youthful facial features is commonly described as the “triangle of youth,” shown in FIG. 2 by the dotted line triangle.

As aging occurs, the triangle becomes referred to as “inverted,” as indicated by the dotted triangle in FIG. 3. The effects of aging are somewhat unique to each individual, but generally, as we age, facial bone changes, soft tissues (like muscles) fall due to gravity, and skin sags and droops downward. Facial fat-pads shift, such as in the cheek area which causes flattened cheeks, while cheekbone loss can reduce previously youthful high cheekbones. Also, fat-pads beneath the chin can increase in prominence, causing fullness between the neck and chin (i.e., “double chin”). This phenomenon of the lower face becoming fuller over time is sometimes described as the “pyramid of age” (see dotted triangle in FIG. 3).

The methods described and contemplated herein focus on aesthetic restoration and reconstruction of facial bones and boney facial features, such as bone deficiency and loss, by administering one or more bone derived compositions by minimally invasive techniques, rather than addressing the more commonly treated age-related soft tissue deficiencies and losses.

The growing social acceptance and desire for gender fluidity and gender reassignment, as well as associated plastic reconstruction and aesthetic procedures, have also created a need for new treatments to adjust body features. It is further contemplated that the methods described and contemplated herein are useful for aesthetic modification of boney features and features associated with facial bones in connection with procedures performed in connection therewith. For example, without limitation, a more defined jawline may be desired to create a more masculine appearance. Soft tissue corrections may not provide the optimum results where a more permanent adjustment of facial features by correction the foundational bone structure and shape may be desired and more effective.

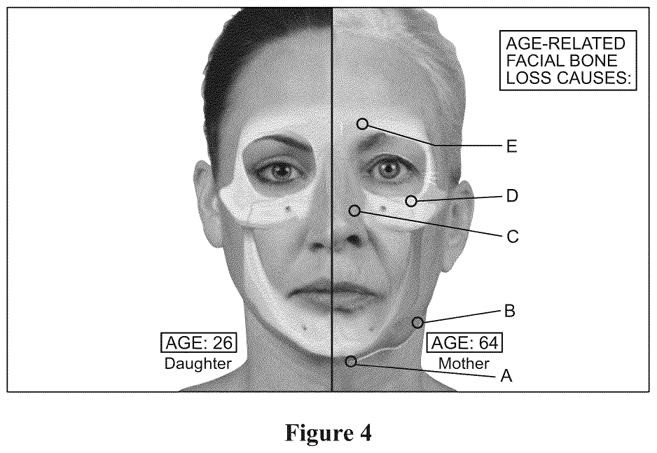

The presently described method for administering bone derived compositions (i.e., bone tissue forms) is targeted at correcting and restoring age-related facial bone loss, such as without limitation, those listed below and shown in FIG. 4 (which is a combination of the images in FIGS. 2 and 3):
A—Loss of chin definition
B—Loss of jawline definition
C—Loss of bone around nasal lines
D—Loss of bone around the lower eye area, resulting in larger eye areas
E—Loss of bone in the upper eye area, which typically results in lowered angle of brow bone, contributing to frown lines, crow's feet and droopy eyelids

According to some exemplary embodiments of the presently described and contemplated method, one or more suitable bone derived compositions are administered (positioned, implanted, etc.) to restore bone defects or loss though a minimally invasive technique (i.e., injection, minimal incision, or both). The selected bone derived compositions may be injected or implanted at any one or more of the aforementioned facial areas to restore bone loss, correct bone defects, and restore volume of the associated facial features. In some embodiments, the bone derived composition(s) may have any one or more preferred, molded, or otherwise formed shapes selected to restore volume, contour, shape, or a combination thereof, when the method is applied to restore a specific facial feature such as chin and jawline definition. Furthermore, the bone derived compositions may retain their original form after implantation, or may solidify or partially solidify in-situ after injection.

It will be understood that the embodiments described herein are merely exemplary and that a person of ordinary skill in the art may make many variations and modifications without departing from the spirit and scope of the invention described and contemplated herein. All such variations and modifications are intended to be included within the scope of the invention described and contemplated herein.

We claim:

1. A method for treatment of a body feature having a condition comprising one or more of bone loss, bone deficit, and bone defect, the method comprising the steps of:
selecting and obtaining one or more bone derived compositions; and
administering the one or more bone derived compositions to a treatment site which is on, in, proximate to, or a combination thereof, the body feature.

2. The method of claim 1, wherein the treatment comprises restoration, reconstruction, volumizing, or both, of one or more of: a size, a shape, a contour, and a volume, of the body feature being treated.

3. The method of claim 1, wherein the step of administering comprises one or more of: implanting, injecting, positioning, reshaping followed by administering, the one or more bone derived compositions to the treatment site, by a minimally invasive procedure.

4. The method of claim 3, wherein the minimally invasive procedure comprises injection using a delivery device, delivery in or through a small incision, or a combination thereof.

5. The method of claim 4, wherein the minimally invasive procedure comprises injection using a delivery device selected from a needle, a syringe, and a cannula, and having a gauge number of about 20 or lower.

6. The method of claim 4, wherein the minimally invasive procedure comprises delivery in or through a small incision having a length from about 1 centimeter to about 0.5 millimeter.

7. The method of claim 1, wherein the one or more bone derived compositions comprise a physical form including: particles, powder, pieces, fibers, at least partially dehydrated, non-dehydrated, flowable, injectable, shapable, at least partially solidifying, preshaped, molded, porous, a paste, a putty, a gel, a suspension, a mixture, a monolithic piece, a mass of smaller forms agglomerated together into a single piece, and combinations thereof.

8. The method of claim 7, wherein the one or more bone derived compositions comprise bone derived fibers.

9. The method of claim 7, wherein the one or more bone derived compositions comprise a demineralized bone matrix (DBM) and are flowable and injectable.

10. The method of claim 9, wherein the one or more bone derived compositions comprise bone derived fibers in a flowable, injectable, composition which further comprises a viscosity modifier comprising hyaluronic acid, derivatives of hyaluronic acid, or both.

11. The method of claim 7, wherein the physical form of the one or more bone derived compositions comprises a monolithic piece, a mass of smaller physical forms agglomerated together into a single piece, or a combination thereof, which has an overall size and shape selected and formed, molded, at least partially solidified, or otherwise produced to be specific to the treatment site or body feature being treated.

12. The method of claim 7, wherein the physical form of the one or more bone derived compositions comprises a porous matrix.

13. The method of claim 1, wherein the one or more bone derived compositions includes one or more properties selected from: containing viable cells, absence of viable cells, containing non-viable cells and cell components, demineralized, partially demineralized, selectively demineralized, non-demineralized, porous, containing one or more endogenous bioactive materials, containing one or more exogenous materials and substances which are bioactive or not.

14. The method of claim 13, wherein the one or more endogenous bioactive materials are selected from: proteins, growth factors, cytokines, vesicles, exosomes, extracellular matrix proteins, cells and cellular components.

15. The method of claim 13, wherein the one or more exogenous materials and substances are selected from: cells, polymers, carriers, excipients, fillers, viscosity modifiers, antibiotics, anticoagulants, and binders.

16. The method of claim 1, wherein the one or more bone derived compositions are combined with other materials and compositions, which are: tissue derived, not tissue derived, endogenous, exogenous, bioactive, inert, natural, synthetic, and combinations thereof.

17. The method of claim 16, wherein the tissue derived materials comprise: cartilage derived materials, placenta derived materials, or a combination thereof.

18. The method of claim 1, wherein the body feature comprises one or more of: a facial feature, a skull, a shoulder, a collarbone, a pelvis, a long bone, a foot, an ankle, a hand, and a wrist.

19. The method of claim 1, wherein the body feature comprises a facial feature which comprises: a jawline, a chin, a brow bone, a cheekbone, a malar area, a bone proximate nasal lines, a bone proximate upper eye area, a bone proximate lower eye area, or a combination thereof.
